(19) United States
(12) Patent Application Publication
(10) Pub. No.: US 2013/0345660 A1
(43) Pub. Date: Dec. 26, 2013

(54) INTRODUCER TACTILE FEATURE

(71) Applicant: Cook Incorporated, Bloomington, IN (US)

(72) Inventors: James J. Kennedy, III, Deerfield, NH (US); Mark R. Frye, Bloomington, IN (US); Bruce Fleck, Anthem, AZ (US)

(73) Assignee: Cook Incorporated, Bloomington, IN (US)

(21) Appl. No.: 14/013,445

(22) Filed: Aug. 29, 2013

Related U.S. Application Data
(63) Continuation of application No. 11/320,041, filed on Dec. 28, 2005.
(60) Provisional application No. 60/640,121, filed on Dec. 29, 2004.

(51) Int. Cl. A61M 25/01

(52) U.S. Cl. CPC A61M 25/0102
USPC 604/500; 604/528

(57) ABSTRACT

An introduction system and method of use is provided. The introduction system includes a catheter assembly, a member, and a tactile indicator. The member is configured to be introduced through the catheter assembly and the tactile indicator is configured to provide a tactile sensory input when the member reaches a predetermined position relative to the catheter assembly. As such, the tactile indicator provides increased resistance to the relative motion between the member and catheter assembly when the member reaches the predetermined position.

### RELATED APPLICATIONS

The present patent document is a continuation of U.S. application Ser. No. 11/320,041, filed Dec. 28, 2005, which claims the benefit of the filing date under 35 U.S.C. §119(e) of Provisional U.S. Patent Application Ser. No. 60/640,121, filed Dec. 29, 2004. All of the foregoing applications are hereby incorporated by reference.

### BACKGROUND

1. Field of the Invention

The present invention generally relates to an introduction system and method to provide sensory input to a user while introducing a medical device.

2. Description of Related Art

Medical devices are typically delivered to the patient through an introduction system. The introduction system typically includes a pre-delivered catheter used to guide the medical device to a region of interest. Often the introduction system will include a sealing adapter to restrict fluid flow from the catheter where the medical device enters the catheter assembly. The user of an introduction system often advances the medical device distally into the patient a predetermined distance through the introduction system. Since the user manually advances the medical device through the introduction system, it is difficult for the user to inherently gauge the distance that the medical device has been advanced. Currently, some introduction systems provide a visual indicator near the proximal end of the introduction system to indicate when the medical device has been advanced a predetermined distance. However, the user must redirect his attention from the patient and any imaging device to the proximal end of the introduction system to observe the visual indicator.

In view of the above, it is apparent that there exists a need for an introduction system for medical device configured to provide improved sensory input to the user.

### SUMMARY

In satisfying the above need, as well as overcoming the enumerated drawbacks and other limitations of the related art, the present invention provides an introduction system for a medical device configured to provide tactile sensory input to the user.

The introduction system includes a catheter assembly, a member, and a tactile indicator. The member is configured to be introduced through the catheter assembly and the tactile indicator is configured to provide a tactile sensory input when the member reaches a predetermined position relative to the catheter assembly. As such, the tactile indicator provides increased resistance to the relative motion between the member and catheter assembly when the member reaches the predetermined position.

The tactile indicator may provide the increased resistance by mechanically interfering with the catheter assembly. Further, the tactile indicator may take the form of a protrusion extending from the member. The protrusion may be created by a bead of solder attached to the member, a prefabricated bead attached to the member, or a bending or folding process that deforms an integral portion of the member. Preferably, the protrusion has rounded edges and may have a generally teardrop shape. Alternatively, the tactile indicator may include a textured portion of the member. The textured portion may have an increased tackiness or surface roughness causing an interference or friction to provide the tactile sensory input.

Further, the tactile indicator may be one of a plurality of tactile indicators. In one respect, the tactile indicator may provide a unique resistance profile with respect to the other tactile indicators. In addition, the frequency between the plurality of tactile indicators may be constant indicating a predefined distance has been traveled or the frequency may be varied along the length of the member to indicate multiple distances or the closeness to a predetermined location.

In another aspect of the invention, the tactile indicator may be a narrowed portion of the catheter assembly that is configured to interfere with the member. Further, the member may include a protrusion to enhance the mechanical interference with the narrowed portion.

In yet another aspect of the present invention, a method for providing sensory input is provided. The method includes introducing a member through a catheter assembly, advancing the member causing a relative motion between the member and the catheter assembly, and increasing a resistance to the relative motion using a tactile indicator to sense a predetermined position of the member relative to the catheter assembly.

Further objects, features and advantages of this invention will become readily apparent to persons skilled in the art after a review of the following description, with reference to the drawings and claims that are appended to and form a part of this specification.

### BRIEF DESCRIPTION OF THE DRAWINGS

FIG. 1 is a breakaway side view of an introduction system having a tactile indicator in accordance with one embodiment of the present invention;

FIG. 2 is a breakaway side view of an introduction system having a tactile indicator attached to an introducer catheter;

FIG. 3A-3E are side views of tactile indicators having varying profiles in accordance with present invention;

FIG. 4 is a breakaway side view of an introduction system having multiple tactile indicators in accordance with one embodiment of the present invention; and

FIG. 5 is a flow chart of a method for using an introduction system in accordance with the present invention.

### DETAILED DESCRIPTION

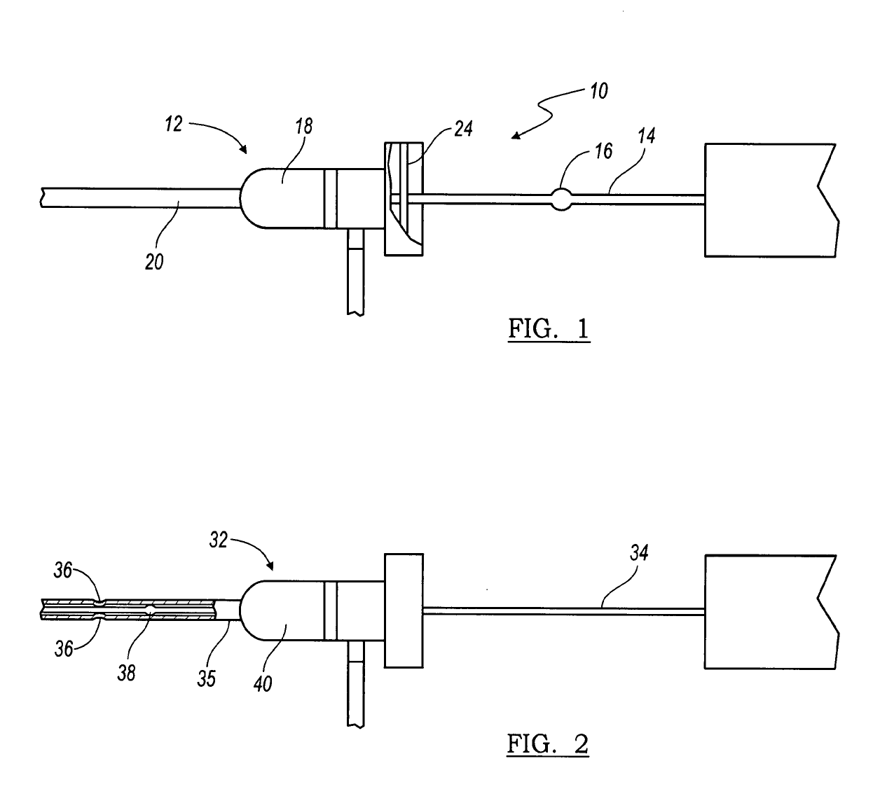

Referring now to FIG. 1, a system embodying the principles of the present invention is illustrated therein and designated at 10. As its primary components, the system 10 includes a catheter assembly 12, a member 14 to be inserted through the catheter assembly 12, and a tactile indicator 16.

The member 14 is shown as an introducer cannula and is inserted into the catheter assembly 12 through a sealing adapter 18. The member 14 is advanced beyond the sealing adapter 18 and through a catheter 20 to a region of interest within the patient. The member 14 includes a tactile indicator 16 shown as a protrusion. The protrusion may be formed by attaching a material to the outer portion of the member 14 at a predetermined position. For example, the member 14 can be made of a solderable material and a bead of silver solder may be applied to the surface of the member 14, thereby forming the tactile indicator 16. Further, a prefabricated bead may be machined or molded and attached to the member 14 via solder, adhesive, or other common attachment means. Alternatively, the member 14 may have a protrusion integrally formed with the member through a bending or forming process.

The sealing adapter may be a Check-Floe type adapter manufactured by Cook Inc., Bloomington, Ind. The sealing adapter 18 includes a seal 24 at its proximal end. The member 14 is inserted into the sealing adapter 18 through the seal 24. As such, the seal 24 acts to restrict fluid from flowing from the catheter assembly 12 as the member 14 is advanced into the patient. When the member 14 is advanced a predetermined distance, a tactile sensory input will be delivered to the user through their fingers. As such, the user will be allowed to maintain focus on the patient or a noninvasive imaging device, rather than focus on a visual indicator at the proximal end of the introduction system 10. The tactile indicator 16 creates a temporary mechanical interference between the member 14 and the seal 24 of the sealing adapter 18. As the member 14 is further advanced, the seal 24 of the sealing adapter 18 will deform to allow the tactile indicator 16 to pass therethrough. Similarly, as the member 14 is withdrawn, the tactile indicator 16 will interfere with the seal 24 of the sealing adapter 18. Again, the user will receive tactile sensory input that the member 14 has been withdrawn to the predefined location.

Now referring to FIG. 2, another embodiment of an introducer system according to the present invention is provided. A member 34 is inserted into a catheter assembly 32 through a sealing adapter 40. The member 34 is advanced beyond the sealing adapter 40 and through catheter 35 to a region of interest within the patient. The catheter 35 may include a tactile indicator 36 shown as a narrowed portion within the catheter 35. The narrowed portion causes a mechanical interference with the member 34 providing the user a tactile sensory input as the member 34 reaches a predetermined location. The diameter of the member 34 may have a profile to vary the mechanical interference and, therefore, the resistance provided by the tactile indicator 36. The tactile indicator 36 may be formed by a reduced diameter of the lumen within the catheter 35, or by other means restricting catheter 35. As the member 34 passes through the tactile indicator 36, the catheter 35 deforms allowing the member 34 to advance along the catheter 35. Similarly, as the member 34 is withdrawn from the catheter assembly 32, the tactile indicator 36 will interfere with the member 34 increasing resistance to the relative motion between the member 34 and the catheter assembly 32. The increased resistance provides a tactile sensory input to the user. Alternatively, the member 34 may include a protrusion 38 to further enhance the tactile sensory input provided to the user as the protrusion 38 passes through the tactile indicator 36 at the predetermined location. As the protrusion 38 passes through the tactile indicator 36, the catheter 35 deforms allowing the protrusion 38 to pass therethrough.

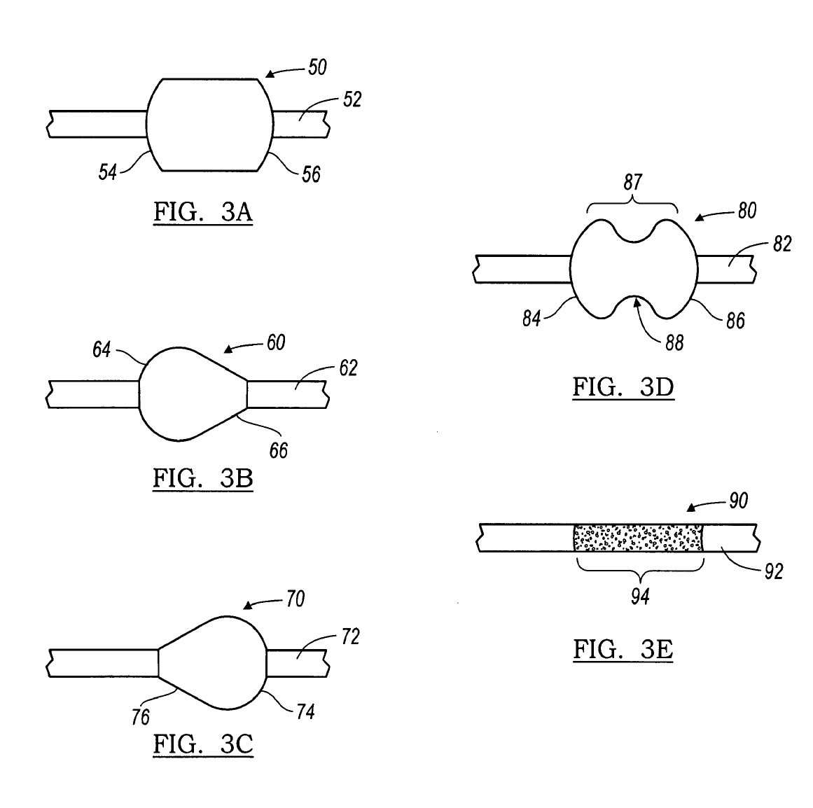

Now referring to FIG. 3-A, a tactile indicator 50 is shown on a member 52, such as, an introduction cannula that may be inserted into a sealing adapter causing a mechanical interference with the seal and providing tactile sensory input for the user. The tactile indicator 50 is shown as a bead attached to the member 52. Preferably, the tactile indicator 50 has a profile that is concentric with the member 52, although asymmetric shapes may be readily used. A concentric profile minimizes and uniformly applies any deformation, caused by the tactile indicator 50, across the circumference of the seal. The tactile indicator 50 has a distal surface 54 that is generally rounded to eliminate any sharp edges. The generally rounded shape of the distal surface 54 prevents damage to the seal as the tactile indicator 50 is advanced therethrough. The slope or curvature of the distal surface 54 may be varied to provide more or less resistance to the advancement of member 52.

The tactile indicator 50 also includes a proximal surface 56 that serves to interfere with the seal of the sealing adapter as the member 52 is withdrawn. The proximal surface 56 has a generally rounded shape to prevent damage of the seal. The slope and curvature of the proximal surface 56 may be varied to provide more or less resistance to the withdrawal of the member 52. In addition, the proximal surface 56 may have a different slope and curvature than the distal surface 54 providing for a different sensory input during advancement and withdrawal of the member 52.

Now referring to FIG. 3-B, a tactile indicator 60 is shown on the member 62, such as, an introduction cannula that may be introduced into a sealing adapter, causing a mechanical interference with the seal. The mechanical interference provides a tactile sensory input for the user. The tactile indicator 60 is shown as a bead attached to the member 62. Preferably, the tactile indicator 60 has a profile that is concentric with the member 62. As such, any deformation of the seal in the sealing adapter caused by the interference of the tactile indicator 60 with the seal will be minimized and uniformly applied across the circumference of the seal. The tactile indicator 60 has a generally teardrop shape, that provides a hard bump or sudden increase in resistance upon insertion, and provides a soft resistance upon removal. The tactile indicator 60 has a distal surface 64 that is generally rounded such that any sharp edges are eliminated to protect the seal during advancement. As such, the distal surface 64 has a slope that is greater than 45° with respect to the surface of the member 62. The distal surface 64 forms the head of the teardrop shape to cause a sudden increase in resistance as the member 62 is advanced to the predetermined position. The proximal surface 66 has a slope that is about 45° or less with respect to the surface of member 62 and forms the tail of the teardrop to cause a soft resistance as the member 62 is withdrawn at the predetermined position.

Alternatively, a teardrop shape may be used to provide soft resistance during insertion and hard resistance during withdraw, as shown in FIG. 3-C. The tactile indicator 70 is shown as a bead attached to the member 72. The proximal surface 76 has a slope that is greater than 45° with respect to the surface of member 72 and forms the tail of the teardrop to cause a soft resistance as the member 72 is advanced to the predetermined position. The distal surface 74 has a slope that is about 45° or less with respect to the surface of the member 72 and forms the head of the teardrop to cause a sudden increase in resistance as the member 72 is withdrawn to the predetermined position.

Now referring to FIG. 3-D, a tactile indicator 80 is shown on the member 82, such as, an introduction cannula that may be introduced into a sealing adapter causing a mechanical interference with the seal and providing tactile sensory input that biases the member 82 to the predetermined position in both a proximal and distal direction. The tactile indicator 80 is shown as a bead attached to the member 82. Preferably, the tactile indicator 80 has a profile that is concentric with the member 82. As such, any deformation of a seal in the sealing adapter, caused by the interference of the tactile indicator 80 with the seal, will be minimized and uniformly applied across the circumference of the seal. The tactile indicator 80 has a distal surface 84 that is generally rounded, such that, any sharp edges are eliminated. The generally rounded shape of the distal surface 84 prevents damage to the seal, as the tactile indicator 80 is advanced therethrough. The tactile indicator 80 also includes a proximal surface 86 that serves to interfere with the seal of the sealing adapter as the member 82 is withdrawn. In addition, the proximal surface 86 may have a different slope and curvature than the distal surface 84 providing for a different sensory input during advancement and withdrawal of the member 82.

Further, a portion 87 of the tactile indicator 80 located between proximal surface 86 in the distal surface 84 may have a profile that varies longitudinally. As such, the portion 87 may narrow in diameter or have a recess 88 that biases the member 82 to the predetermined location in both a distal and proximal direction. The recess 88 has two surfaces sloped towards each other such that when the member 82 is in the predetermined location the seal aligns with the recess 88. The proximal most surface of the recess 88 biases the seal distally and a distal most surface of the recess 88 biases the seal proximally. The slope of the recess 88 and the length of the portion 87 can be determined, such that, both surfaces of the recess 88 simultaneously engage seal.

Now referring to FIG. 3-E, a tactile indicator 90 is shown on the member 92 such as, an introduction cannula. The tactile indicator 90 shown as a textured portion 94 on the surface of the member 92. The member 92 may be introduced into a sealing adapter causing interference and friction between the seal of the sealing adapter and the tactile indicator 90. The interference provides tactile sensory input for the user. The textured portion 94 may include a coating to increase the tackiness or surface roughness of the textured portion 94 with respect to the rest of the surface of member 92. Alternatively, a coating may be omitted from the textured portion 94 that has been applied to the rest of the surface of member 92 thereby increasing the tackiness or surface roughness of the textured portion 94 relative to other portions of member 92.

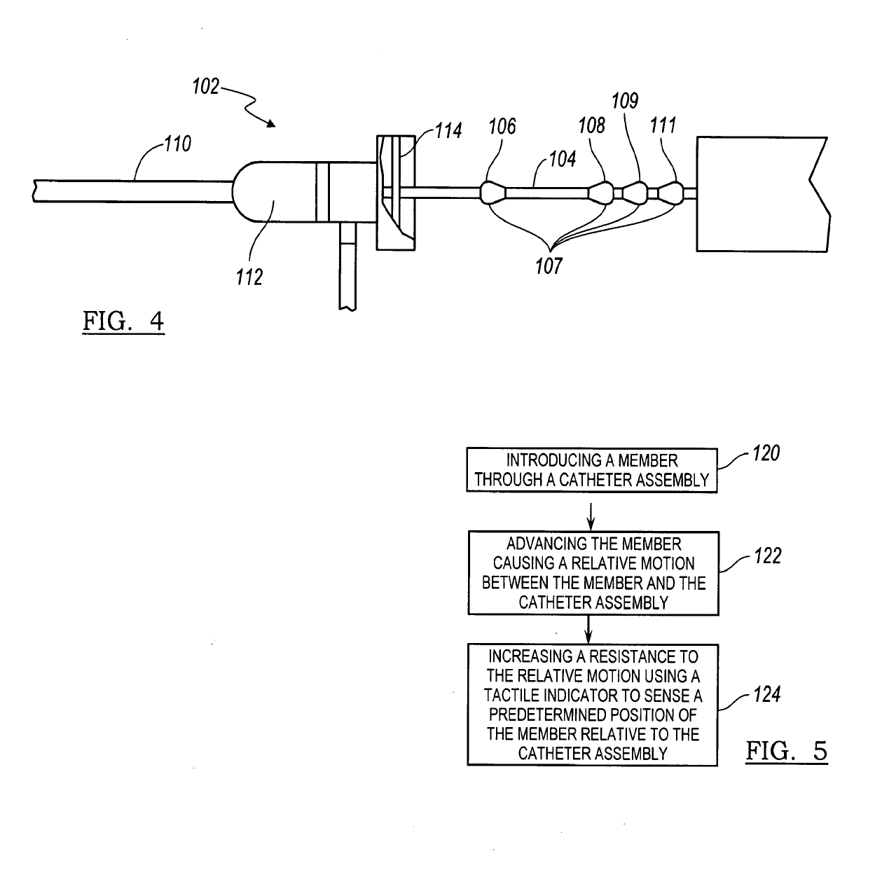

Now referring to FIG. 4, an introduction system is provided including a catheter assembly 102, a member 104 to be inserted through the catheter assembly 102, and a plurality of tactile indicators 107. The member 104 is shown as an introducer cannula and is inserted into the catheter assembly 102 through a sealing adapter 112. The member 104 is advanced beyond the sealing adapter 112 and through a catheter 110 to a region of interest within the patient. The member 104 includes tactile indicators 107, shown as beads attached to the surface of the member 104. Each tactile indicator 106, 108, 109, and 111 is located at a predetermined position along the member 104. Further, tactile indicator 106 has a different longitudinal profile than tactile indicator 108. For example, tactile indicator 106 has a teardrop shape with the head of the teardrop facing distally, while tactile indicators 108, 109, and 111 have a teardrop shape with the head facing proximally. The frequency between beads may also be varied. For example, a first resistance is felt when tactile indicator 106 passes through the seal 114. As the member 104 is advanced, the user will feel resistances at an increased frequency as tactile indicators 108 and 109 pass through the seal 114. The increased frequency may indicate the user is approaching a predetermined location, or may have other preferred significance. Alternatively, the frequency of the tactile indicators may be constant along the length of the member 114 indicating a predetermined distance has been traveled between the tactile indicators, such as between tactile indicators 108, 109, and 111.

The member 104 is inserted into the sealing adapter 112 through a seal 114. As such, the seal 114 acts to restrict fluid from flowing from the catheter assembly 102 as the member 104 is advanced into the patient. When the member 104 is advanced a predetermined distance, a tactile sensory input will be delivered to the user, through their fingers. The tactile indicator 106 creates a temporary mechanical interference between the head of tactile indicator 106 and the seal 114 of the sealing adapter 112, causing a sudden increase in resistance. As the member 104 is further advanced, the seal 114 of the sealing adapter 112 will deform to allow tactile indicator 106 to pass therethrough. As tactile indicator 108 is advanced through the seal 114 the tail of indicator 108 causes a soft resistance easily distinguishable from tactile indicator 106. Similarly, as the member 104 is withdrawn, the head of tactile indicator 108 will interfere with the seal 114 of the sealing adapter 112. Again the user will receive tactile sensory input that the member 104 is being withdrawn to a predefined location. As member 104 is withdrawn further, the tail of tactile indicator 106 will cause a soft resistance as the seal 114 is deformed, allowing tactile indicator 106 to pass therethrough. The opposing teardrop configuration discussed is merely illustrative and many combinations of tactile indicator profiles are contemplated herein.

Now referring to FIG. 5, a method is provided for generating a tactile sensory input for the user of an introduction system. The method includes introducing a member through a catheter assembly as denoted by block 120. The member is advanced into the patient causing a relative motion between the member and the catheter assembly as denoted by block 122. As the member is advanced, a tactile indicator is used to increase a resistance to the relative motion when the member reaches a predetermined position relative to the catheter assembly as denoted by block 124. The method described herein may be implemented according to any of the variations or embodiments described above.

As a person skilled in the art will readily appreciate, the above description is meant as an illustration of implementation of the principles this invention. This description is not intended to limit the scope or application of this invention in that the invention is susceptible to modification, variation and change, without departing from spirit of this invention, as defined in the following claims.

I/we claim:

1. An introduction system for a medical device, the introduction system comprising:
a catheter assembly;
a member to be introduced through the catheter assembly; and a tactile indicator configured to provide a tactile sensory input when the member reaches a predetermined position relative to the catheter assembly.

2. The introduction system according to claim 1, wherein the tactile indicator provides increased resistance to a relative motion between the member and the catheter assembly when the member reaches the predetermined position.

3. The introduction system according to claim 1, wherein the tactile indicator mechanically interferes with the catheter assembly to provide the tactile sensory input.

4. The introduction system according to claim 1, wherein the tactile indicator comprises a protrusion extending from the member.

5. The introduction system according to claim 4, wherein the tactile indicator comprises a bead attached to the member.

6. The introduction system according to claim 4, wherein the protrusion has rounded edges.

7. The introduction system according to claim 4, wherein the protrusion has a generally teardrop shape.

8. The introduction system according to claim 4, wherein the protrusion includes a first surface forming an angle greater than 45° with the member.

9. The introduction system according to claim 8, wherein the protrusion includes a second surface forming an angle about 45° or less with respect to the member.

10. The introduction system according to claim 1, wherein the catheter assembly includes a sealing adapter.

11. The introduction system according to claim 10, wherein the tactile indicator mechanically interferes with the sealing adapter.

12. The introduction system according to claim 1, wherein the tactile indicator includes a textured portion of the member.

13. The introduction system according to claim 12, wherein the textured portion has an increased surface roughness.

14. The introduction system according to claim 12, wherein the member includes a coating and the coating has been omitted from the textured portion.

15. The introduction system according to claim 1, wherein the tactile indicator is one of a plurality of tactile indicators.

16. The introduction system according to claim 15, wherein the tactile indicator provides a differentiating resistance profile with respect to the plurality of tactile indicators.

17. The introduction system according to claim 15, wherein the frequency of the plurality of tactile indicators is varied longitudinally along the member.

18. The introduction system according to claim 15, wherein the frequency of the plurality of tactile indicators is constant.

19. The introduction system according to claim 1, wherein the catheter assembly includes a catheter and the tactile indicator includes a narrowed portion of the catheter configured to interfere with the member.

20. The introduction system according to claim 19, wherein the member includes a protrusion that interferes with the narrowed portion.

21. The introduction system according to claim 1, wherein the tactile indicator includes a protrusion extending from the member, the protrusion having a distal surface and a proximal surface, and the protrusion including a recess between the proximal and distal surface.

22. The introduction system according to claim 21, wherein the recess is configured to bias the member in both a distal and proximal direction toward the predetermined location.

23. A method of providing a tactile sensory input to the user of an introduction system, the method comprising:
introducing a member through a catheter assembly;
advancing the member causing a relative motion between the member and the catheter assembly;
increasing a resistance to the relative motion using a tactile indicator to sense a predetermined position of the member relative to the catheter assembly.

24. The method according to claim 23, wherein the tactile indicator mechanically interferes with the catheter assembly to provide the tactile sensory input.

25. The method according to claim 23, wherein the tactile indicator comprises a protrusion extending from the member.
